(19) United States
(12) Patent Application Publication
(10) Pub. No.: US 2007/0078419 A1
(43) Pub. Date: Apr. 5, 2007

(54) Changing table cover

(76) Inventor: Barbara Bredfeldt, Tustin, CA (US)

Correspondence Address: John H. Lynn, Suite C 103, 2915 Redhill Avenue, Costa Mesa, CA, 92626, US

(21) Appl. No.: 11/243,572

(22) Filed: Oct. 4, 2005

(51) Int. Cl. A61B 19/00

(52) U.S. Cl. 604/357

(57) ABSTRACT

A changing table cover for providing a sanitary environment for changing an article of clothing worn by a child comprises a sheet of flexible material formed to have a lower layer formed of a waterproof material and an upper layer bonded to the lower layer. The table cover comprises a mounting device attached to the lower layer and arranged for mounting the sheet of flexible material to a table to cover at least a portion of the tabletop so that a child may be placed on the second layer without coming into direct contact with the tabletop. The mounting device may comprise an adhesive attached to the lower layer and arranged to temporarily adhere to the tabletop and to permit easy removal therefrom after the sheet is no longer needed to provide a sanitary environment. The mounting device may alternatively comprise an elastic band connected to the lower layer.

### BACKGROUND OF THE INVENTION

This invention relates generally to table covers and particularly to a cover for diaper changing tables that are is found in many public places.

The restrooms in many public places have tables available for a person taking care of a small child to use for purposes such as attending to the child's hygienic needs or changing an article of the child's clothing. Concerns have arisen regarding the sanitation of such facilities, particularly those found in public restrooms. It is possible to use a towel or a sheet of paper or plastic to cover a changing table. However, the items that are typically available are often too small to cover the entire table top and have no means for keeping them in place between a squirming child and the tabletop with suspect sanitary conditions.

Even when there is no facility available that is specifically designed or dedicated to use as a changing station, it is desirable to have an easy to use, sanitary disposable cover to place between a child whose diaper is being changed and whatever surface is available when the need arises.

### SUMMARY OF THE INVENTION

Accordingly, the present invention provides a changing table cover that may be secured to a table and that covers an area adequate to prevent both the child and the caregiver from contacting the tabletop, and thereby from having direct contact with bacteria, viruses and other contaminants that might be on the table.

A table cover according to the present invention for placement on a tabletop to provide a sanitary environment for changing or adjusting an article of clothing worn by a child comprises a sheet of flexible material formed to have a lower layer formed of a waterproof material and an upper layer bonded to the lower layer. The table cover further comprises a mounting device attached to the lower layer and arranged for mounting the sheet of flexible material to a table to cover at least a portion of the tabletop so that a child may be placed on the second layer without coming into direct contact with the tabletop. The mounting device may comprise an adhesive attached to the lower layer with the adhesive being formed to temporarily adhere to the tabletop and to permit easy removal therefrom after the sheet is no longer needed to provide a sanitary environment.

The mounting device may alternatively comprise an elastic band connected adjacent an edge portion of the lower layer.

The advantages of the invention and its structural features may be understood and appreciated by referring to the drawings and to the detailed description of the invention. It should be understood that none of the features illustrated in the drawings are drawn to any scale. Instead, the dimensions of certain features are exaggerated in the interest of clarity.

### BRIEF DESCRIPTION OF THE DRAWINGS

FIG. 1 is a perspective view of a table cover according to the present invention being used to cover a table top;

FIG. 2 is a top plan view of the table cover of FIG. 1;

FIG. 3 is end elevation view of the table cover of FIGS. 1 and 2;

FIG. 4 is a perspective view showing table covers according to the present invention formed in a roll to facilitate storage and dispensing;

FIG. 5 is a perspective view of an alternate embodiment of the invention that includes an elastic band being used to secure a table cover to a tabletop;

FIG. 6 is a partial cross sectional view of the embodiment of the invention shown in FIG. 5 showing an elastic band mounted in an inner side portion; and

FIG. 7 is a cross sectional view showing the elastic band of FIG. 6 mounted inside a sleeve attached to the table cover

### DETAILED DESCRIPTION OF THE INVENTION

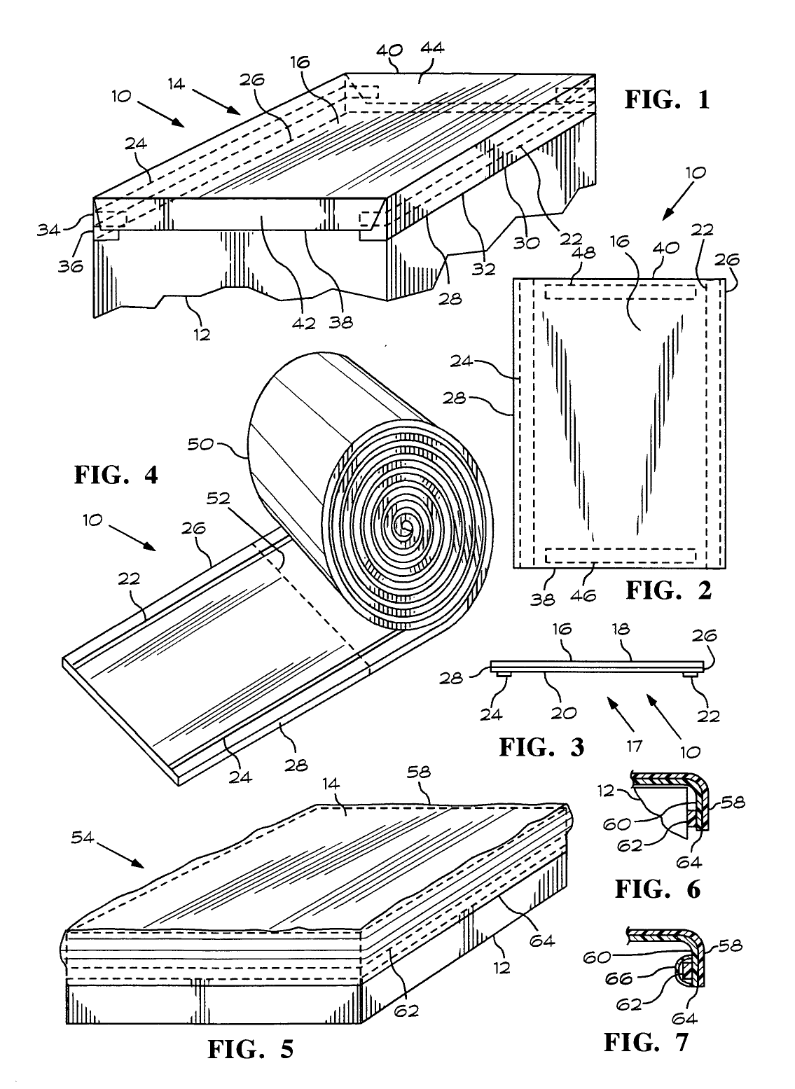

Referring to FIG. 1, a table cover 10 according to the present invention is attached to a table 12 and arranged to cover a tabletop 14. Referring also to FIGS. 2 and 3, the table cover 10 may be formed to have a rectangular upper surface 16. The upper surface 16 preferably is about 36 inches in length and about 20 inches in width. These dimensions are adequate to cover the tops of changing table typically found in public restrooms.

As shown in the end elevation view of FIG. 3, the table cover 10 preferably is formed as a sheet 17 of material having an upper layer 18 and a lower layer 20 that are bonded together. The upper layer 18 may be formed of a material such as a light-weight paper or a suitable fabric. The lower layer 20 preferably is formed of a waterproof material such as a thin sheet of plastic.

Still referring to FIGS. 1-3, a pair of adhesive strips 22 and 24 is attached to the lower layer 20. The adhesive strips 22 and 24 preferably are parallel and located near opposite side edges 26 and 28, respectively, of the table cover 10. As shown in FIG. 1, the dimensions of the table cover 10 preferably are greater than the corresponding dimensions of the tabletop 14. The table cover 10 preferably is placed on the tabletop 14 with a side portion 30 of the table cover 10 being folded over a side portion 32 of the table 12. The adhesive strip 22 preferably is located near the edge 28 of the table cover 28 so that the adhesive strip 22 may be adhered to the side portion 32 of the table 12.

A side portion 34 of the table cover 10 preferably extends over a side portion 36 of the table 12 so that the adhesive strip 24 may be adhered to a corresponding side portion 36 of the table 12. End portions 38 and 40 are preferably folded over corresponding end portions 42 and 44 of the table 12.

As shown in FIG. 2, the table cover 10 may optionally include adhesive strips 46 and 48 attached to the end portions 38 and 40 to provide means for securing the table cover 10 to the end portions 42 and 44 of the table 12.

It is therefore readily seen that in the embodiment of the invention shown in FIGS. 1-3 and described above, the table cover 10 may be used to cover the entire tabletop 14. However, the invention is not limited in its utility to application to tabletops that may be completely covered. For example, if a tabletop (not shown) has a width greater than the width of the table cover 10, then the table cover 10 may be positioned such that the side portion 30 (for example) is folded over one side portion of the table and adhered to the side portion as shown in FIG. 1. The other side portion 34 will then lay flat upon the tabletop 14 with the adhesive strip 24 adhered thereto. If the length of the table is greater than the length of the table cover 10, then on or both of the end portions 38 and 40 of the table cover 10 will be flat upon the table.

The table cover 10 may be packaged as sheets that may be either flat or folded. Alternatively, a roll 50 of a long sheet of table covers 10 according to the present invention may be formed as shown in FIG. 4. The individual table covers 10 may be separated by perforations 52 in the roll 50.

FIGS. 5-7 illustrate an alternate embodiment of the invention. A table cover 54 is formed to have a generally rectangular configuration and preferably has upper and lower layers 58 and 60, respectively, similar to the layers 18 and 20 as described with reference to FIG. 2. An elastic band 62 is mounted to the table cover 54 adjacent an edge portion 64. The elastic band 62 preferably extends completely around the perimeter of the table cover 54. The unstretched length of the elastic band 62 is preferably less than the length of the perimeter of the tabletop 14. Applying the table cover 54 to the tabletop 14 therefore involves stretching the elastic band 62 an amount sufficient to place it around the perimeter of the tabletop 14 at the upper edge portion 64.

The elastic band 62 may be bonded to the lower layer 60, or it may be mounted to the lower layer 62 by any other convenient means. For example, the elastic band 62 may be placed inside a sleeve 66 that extends around the outer edge portion as indicated in FIG. 7.

What is claimed is:

1. A table cover for placement on a tabletop to provide a sanitary environment for changing or adjusting an article of clothing worn by a child, comprising:
a sheet of flexible material having a first side and a second side;
mounting means attached to the first side such for connecting the sheet of flexible material to a table to cover at least a portion of the tabletop so that a child may be placed on the second layer without coming into direct contact with the tabletop.

2. The table cover of claim 1 wherein the mounting means comprises an adhesive attached to the first side of the sheet with the adhesive being formed to temporarily adhere to the tabletop and to permit easy removal therefrom after the sheet is no longer needed to provide a sanitary environment.

3. The table cover of claim 2 wherein the sheet is formed as a multilayer structure having a lower layer formed of a waterproof material.

4. The table cover of claim 2 wherein the sheet comprises a lower layer formed of a waterproof material and an upper sheet formed of paper.

5. The table cover of claim 2 wherein the sheet comprises a lower layer formed of a waterproof material and an upper sheet formed of a fabric.

6. The table cover of claim 2 wherein the adhesive is formed to comprise a plurality of adhesive strips attached to the first side of the sheet.

7. The table cover of claim 6 wherein the sheet is formed as a multilayer structure having a lower layer formed of a waterproof material.

8. The table cover of claim 6 wherein the sheet comprises a lower layer formed of a waterproof material and an upper sheet formed of paper.

9. The table cover of claim 6 wherein the sheet comprises a lower layer formed of a waterproof material and an upper sheet formed of a fabric.

10. The table cover of claim 1 wherein a plurality of the sheets are connected end-to-end and rolled into a generally cylindrical form with perforations between adjacent sheets so that they may be easily separated.

11. The table cover of claim 1 wherein the mounting means comprises an elastic band connected adjacent an edge portion of the first side of the sheet.

12. The table cover of claim 11 wherein the sheet is formed as a multilayer structure having a lower layer formed of a waterproof material.

13. The table cover of claim 11 wherein the sheet comprises a lower layer formed of a waterproof material and an upper sheet formed of paper.

14. The table cover of claim 11 wherein the sheet comprises a lower layer formed of a waterproof material and an upper sheet formed of a fabric.

15. A table cover for placement on a tabletop to provide a sanitary environment for changing or adjusting an article of clothing worn by a child, comprising:
a sheet of flexible material formed to have a lower layer formed of a waterproof material and an upper layer bonded to the lower layer;
a mounting device attached to the lower layer and arranged for mounting the sheet of flexible material to a table to cover at least a portion of the tabletop so that a child may be placed on the second layer without coming into direct contact with the tabletop.

16. The table cover of claim 15 wherein the mounting device comprises an adhesive attached to the lower layer with the adhesive being formed to temporarily adhere to the tabletop and to permit easy removal therefrom after the sheet is no longer needed to provide a sanitary environment.

17. The table cover of claim 16 wherein the mounting device comprises an elastic band connected adjacent an edge portion of the lower layer.
